SEQUENCE LISTING (1 sequence)

SEQ ID NO: 1 - DNA, 1291 bp, Artificial Sequence
  Proteus mirabilis
gaaacggtggctaataccgcataatgtctacggaccaaagcaggggctcttcggaccttgcactatcggatgaacccatatgggattagctagtaggtggggtaaaggctcacctaggcgacgatctctagctggtctgagaggatgatcagccacactgggactgagacacggcccagactcctacgggaggcagcagtggggaatattgcacaatgggcgcaagcctgatgcagccatgccgcgtgtatgaagaaggccttagggttgtaaagtactttcagcggggaggaaggtgataaggttaatacccttgtcaattgacgttacccgcagatgaagcaccggctaactccgtgccagcagccgcggtaatacggagggtgcaagcgttaatcggaattactgggcgtaaagcgcacgcaggcggtcaattaagtcagatgtgaaagccccgagcttaacttgggaattgcatctgaaactggttggctagagtcttgtagaggggggtagaattccatgtgtagcggtgaaatgcgtagagatgtggaggaataccggtggcgaaggcggccccctggacaaagactgacgctcaggtgcgaaagcgtggggagcaaacaggattagataccctggtagtccacgctgtaaacgatgtcgatttagaggttgtggtcttgaaccgtggcttctggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagcgaatcctttagagatagaggagtgccttcgggaacgctgagacaggtgctgcatggctgtcgtcagctcgtgttgtgaaatgttgggttaagtcccgcaacgagcgcaacccttatcctttgttgccagcacgtaatggtgggaactcaaaggagactgccggtgataaaccggaggaaggtggggatgacgtcaagtcatcatggcccttacgagtagggctacacacgtgctacaatggcagatacaaagagaagcgacctcgcgagagcaagcggaactcataaagtctgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaac

(19) United States
(12) Patent Application Publication
(10) Pub. No.: US 2020/0128801 A1
(43) Pub. Date: Apr. 30, 2020

(54) INFORMATION PROVIDING METHOD FOR DIAGNOSING PARKINSONS DISEASE

(71) Applicant: UNIVERSITY-INDUSTRY COOPERATION GROUP OF KYUNG HEE UNIVERSITY, Yongin-si, Gyeonggi-do (KR)

(72) Inventors: Myung Sook OH, Seoul (KR); Dong-Hyun KIM, Seoul (KR); Jin Gyu CHOI, Seoul (KR)

(21) Appl. No.: 16/467,350

(22) PCT Filed: Dec. 6, 2017
(86) PCT No.: PCT/KR2017/014239

§ 371 Date: Jun. 6, 2019

(30) Foreign Application Priority Data
Dec. 6, 2016 (KR) 10-2016-0165136

(51) Int. Cl. A01K 67/027; C12Q 1/06; G01N 33/68; A61K 49/00

(52) U.S. Cl. CPC A01K 67/027; C12Q 1/06; G01N 33/6896; A61K 49/0008; A01K 2267/0318; G01N 2400/50; A01K 2207/20; A01K 2207/25; G01N 2333/24

(57) ABSTRACT

Provided is an information providing method for diagnosing Parkinson's disease by measuring the amount of any one target selected from the group consisting of a Proteus mirabilis strain, a metabolite produced by the Proteus mirabilis strain, and α-synuclein in a biological sample of a subject; a composition comprising a Proteus mirabilis strain as an active ingredient for fabricating a Parkinson's disease animal model; a method for fabricating a Parkinson's disease animal model, the method comprising a step for administering a Proteus mirabilis strain to an animal excluding human; and a method for screening a Parkinson's disease medicine, the method comprising a step for administering a candidate drug of the Parkinson's disease medicine to the Parkinson's disease animal model and a step for observing the degree of mitigation of Parkinson's disease symptoms to determine the treatment effect of the candidate drug on Parkinson's disease.

### TECHNICAL FIELD

The present invention relates to an information providing method for diagnosing Parkinson's disease by measuring the amount of any one target selected from the group consisting of a Proteus mirabilis strain, a metabolite produced by the Proteus mirabilis strain, and α-synuclein in a biological sample of a subject; a composition for fabricating a Parkinson's disease animal model, comprising a Proteus mirabilis strain as an active ingredient; a method for fabricating a Parkinson's disease animal model, comprising administering a Proteus mirabilis strain to an animal excluding humans; and a method for screening an agent for treating Parkinson's disease, comprising administering a candidate drug of the agent for treating Parkinson's disease to the Parkinson's disease animal model and observing the degree of mitigation of Parkinson's disease symptoms to determine the treatment effect of the candidate drug on Parkinson's disease.

### BACKGROUND ART

Parkinson's disease (PD) is a disease which has tremor, rigidity, bradykinesia, gait abnormality, etc. as its main symptoms. Parkinson's disease is a chronic disease caused by the lack of a neurotransmitter referred to as dopamine at the site of the substantia nigra and the corpus striatum of the brain. The dopamine is a substance that is produced at the site of the substantia nigra of the brain and secreted to control the function of basal ganglia, which is a very important site in controlling motor functions.

In other words, Parkinson's disease occurs due to the lack of dopamine which controls the function of the basal ganglia when the substantia nigra of the brain is damaged. Currently, drug therapy, surgical therapy, physical therapy, etc. are used as a method for treating Parkinson's disease.

As for drug therapy, in general, the therapy aims at supplementing the dopamine shortage in the brain, correcting the imbalance of neurotransmitters caused by the dopamine shortage, preventing or delaying the damage to neurons, and administering drugs to control other symptoms such as depression, etc.

However, when the major symptoms of Parkinson's disease are initially expressed and a patient is diagnosed with Parkinson's disease, in general, already 70% or more of the dopaminergic neurons in the substantia nigra is damaged. Since neurons already dead cannot be revived, the drugs do not aim at full recovery, but merely aim at improvement of the symptoms. Therefore, there are limitations in treating Parkinson's disease by drug therapy.

Meanwhile, the extended excretion time of the gastrointestinal tract or constipation is a symptom most of the patients experience at an early stage of Parkinson's disease. Accordingly, the research on relationship between Parkinson's disease and intestinal functions may provide information useful for developing technology to investigate the cause of the disease and provide early diagnosis.

Conventionally, it has been known that the brain and intestines are closely connected to each other for interaction and that intestinal microorganisms existing in the intestines maintain the homoeostasis of the intestinal environment and are involved in the production of neurotransmitters in the brain. In particular, it has been known that the change in intestinal microorganisms is associated not only with metabolic diseases such as obesity, diabetics, etc., but also with mental illnesses such as depression, autism, etc. and degenerative brain diseases such as Alzheimer's disease, Parkinson's disease, etc.

There have been clinical reports on correlation between the increase of Enterobacteriaceae family strains in the feces of a Parkinson's disease patient and Parkinson's disease, but there has been no report on a research providing experimental identification.

In this regard, while conducting a research on correlation between intestinal microorganisms and Parkinson's disease, the present inventors found out for the first time that a Proteus mirabilis strain exists in Parkinson's disease animal models and the Proteus mirabilis strain is directly involved in the occurrence of Parkinson's disease, and completed the present invention.

### DISCLOSURE

### Technical Problem

It is an object of the present invention to provide an information providing method for diagnosing Parkinson's disease, comprising a) measuring the amount of any one target selected from the group consisting of a Proteus mirabilis strain, a metabolite produced by the Proteus mirabilis strain, and α-synuclein in a biological sample of a subject; and b) comparing the amount of the target measured in the above step a) with the amount of the target measured from a biological sample of a normal control group which is not suffering from Parkinson's disease.

It is another object of the present invention to provide a composition for fabricating a Parkinson's disease animal model, comprising a Proteus mirabilis strain as an active ingredient.

It is yet another object of the present invention to provide a method for fabricating a Parkinson's disease animal model, comprising administering a Proteus mirabilis strain to an animal excluding humans.

It is yet another object of the present invention to provide a method for screening an agent for treating Parkinson's disease, comprising administering a candidate drug of the agent for treating Parkinson's disease to the Parkinson's disease animal model fabricated; and observing the degree of mitigation of Parkinson's disease symptoms to determine the treatment effect of the candidate drug on Parkinson's disease.

### Technical Solution

Hereinafter, the present invention will be explained in detail.

According to an embodiment of the present invention, the present invention provides an information providing method for diagnosing Parkinson's disease, comprising: a) measuring the amount of any one target selected from the group consisting of a Proteus mirabilis strain, a metabolite produced by the Proteus mirabilis strain, and α-synuclein in a biological sample of a subject; and b) comparing the amount of the target measured in the above step a) with the amount of the target measured from a biological sample of a normal control group which is not suffering from Parkinson's disease.

Preferably, the information providing method for diagnosing Parkinson's disease further comprises c) classifying, i) when the amount of the target measured in the above step a) is greater than the amount of the target measured from a biological sample of a normal control group which is not suffering from Parkinson's disease, that the subject develops or has a high risk of developing Parkinson's disease, and classifying, ii) when the amount of the target measured in the above step a) is similar or equal to the amount of the target measured from a biological sample of a normal control group which is not suffering from Parkinson's disease, that the subject does not develop Parkinson's disease.

Step a) in the information providing method of the present invention is a step of measuring the amount of any one target selected from the group consisting of a Proteus mirabilis strain, a metabolite produced by the Proteus mirabilis strain, and α-synuclein in a biological sample of a subject.

In the present invention, the subject means a living organism used for testing, examining, analyzing, evaluating, etc. Preferably, the subject may be mammals (e.g., humans, monkeys, cows, horses, rats, mice, guinea pigs, rabbits, dogs, cats, sheep, goats, etc.), and more preferably, the subject may be humans.

In the present invention, the biological sample refers to a sample collected from a subject where the amount of any one target selected from the group consisting of a Proteus mirabilis strain, a metabolite produced by the Proteus mirabilis strain, and α-synuclein is different from the amount of the normal control group according to the occurrence or the progress of Parkinson's disease.

The sample may include samples such as tissue, blood, whole blood, serum, plasma, saliva, sputum, cerebrospinal fluid, urine, colonic tissue or feces of the subject, but is not limited thereto. Preferably, the sample may be colonic tissue or feces. The biological sample may be obtained by a method not harming the subject.

The biological sample of the subject may be a sample separated from a living body.

The Proteus mirabilis strain is a gram negative rod-shaped stain which belongs to the Enterobacteriaceae family. The Proteus mirabilis strain is known as a strain biosynthesizing lipopolysaccharide (LPS) which causes inflammation.

The present invention confirmed the use of the Proteus mirabilis strain as a biomarker for examining the occurrence of Parkinson's disease by separating the Proteus mirabilis strain from the feces of various Parkinson's disease animal models, and identifying the same.

In a specific example of the present invention, as a result of measuring the number of bacteria of the Proteus mirabilis strain and the number of bacteria of other strains of the Enterobacteriaceae family to which the Proteus mirabilis strain belongs, i.e., E. coli and Klebsiella sp. strains, in an MPTP/Proteus mirabilis strain induced Parkinson's disease animal model, and comparing the number with that of a normal control group, the number of bacteria of the Proteus mirabilis strain was remarkably greater than that of the normal control group, as compared with the two strains (FIG. 9).

In the present invention, measuring the amount of Proteus mirabilis strain means measuring the number of Proteus mirabilis strain in a biological sample of a subject. The measurement may be performed using direct microscopic count method, optical density (OD) method or colony count method expressed by colony forming unit (CFU), etc., but the measurement may be performed by any method known to a person skilled in the art without limitation. In the present invention, preferably, the method for measuring the amount of Proteus mirabilis strain is the colony count method.

The metabolite produced by the Proteus mirabilis strain refers to an intermediate product produced during the metabolic process of the Proteus mirabilis strain or a product. Preferably, the metabolite may be lipopolysaccharide (LPS) produced by the Proteus mirabilis strain.

In the present invention, the amount of lipopolysaccharide may be measured by any method known to a person skilled in the art without limitation, and preferably, the amount of lipopolysaccharide may be measured using a kit for quantification.

The α-synuclein is a protein helping neural transmission between brain cells, and the overexpression of this protein is known as a major cause of Parkinson's disease.

In the present invention, measuring the amount of α-synuclein means measuring the expression level of α-synuclein in a biological sample of a subject. The measurement may be performed using western blot, enzyme linked immunosorbent assay (ELISA), immunohistochemisty, immunoprecipitation assay and fluorescence activated cell sorter (FACS), etc., but the measurement may be performed by any method known to a person skilled in the art without limitation. In the present invention, preferably, the method for measuring the amount of α-synuclein is western blot.

Step b) in the information providing method of the present invention is a step of comparing the amount of the target measured in the above step a) with the amount of the target measured from a biological sample of a normal control group which is not suffering from Parkinson's disease.

The normal control group not suffering from Parkinson's disease is a counterpart of the aforementioned subject showing symptoms of Parkinson's disease, which is the target for comparison. Specifically, the normal control group is a subject not suffering from Parkinson's disease, which means a living organism not suffering from Parkinson's disease, used for testing, examining, analyzing, evaluating, etc. Preferably, the subject may be mammals (e.g., humans, monkeys, cows, horses, rats, mice, guinea pigs, rabbits, dogs, cats, sheep, goats, etc.) not suffering from Parkinson's disease, and more preferably, the subject may be humans.

Step c) in the information providing method of the present invention is a step of classifying, i) when the amount of the target measured in the above step a) is greater than the amount of the target measured from a biological sample of a normal control group which is not suffering from Parkinson's disease, that the subject develops Parkinson's disease, and classifying, ii) when the amount of the target measured in the above step a) is similar or equal to the amount of the target measured from a biological sample of a normal control group which is not suffering from Parkinson's disease, that the subject develops or has a high risk of developing Parkinson's disease. This step provides information for diagnosing Parkinson's disease.

In the present invention, diagnosing means confirming the presence or features of pathological conditions. According to the object of the present invention, diagnosing means confirming whether Parkinson's disease occurs.

In a specific example of the present invention, it has been confirmed that the number of bacteria of the Proteus mirabilis strain in an MPTP-induced Parkinson's disease animal model is about 12 times greater than that of the normal control group (FIG. 9), and the number of bacteria of the Proteus mirabilis strain in an MPTP/Proteus mirabilis strain-induced Parkinson's disease animal model is about 15 times greater than that of the normal control group (FIG. 9).

Also, in a specific example of the present invention, it has been confirmed that after administering the Proteus mirabilis strain to a Parkinson's disease animal model by the Proteus mirabilis strain, over time, the α-synuclein level statistically significantly increased on the 16^{th} day in the large intestine and on the 32^{nd} day in the substantia nigra of the brain after administering the Proteus mirabilis strain, as compared with the normal control group (FIG. 5).

Also, in a specific example of the present invention, it has been confirmed that after administering the Proteus mirabilis strain to a Proteus mirabilis strain induced Parkinson's disease animal model, over time. LPS increased statistically significantly on the 1^{st}, 8^{th} and 16^{th} day in the feces and on the 16^{th} and 32^{nd} day in the plasma after administering the Proteus mirabilis strain, as compared with the normal control group (FIG. 4).

According to another embodiment of the present invention, the present invention provides a composition for fabricating a Parkinson's disease animal model, comprising a Proteus mirabilis strain as an active ingredient.

In general, a Parkinson's disease animal model is fabricated by administering, to a test animal, catecholamine neurotoxin 6-OHDA (6-hydroxydopamine) which shows neurotoxin selectively to dopaminergic neurons, MPTP (1-methyl-4-phenyl-1,2,4,6-tetrahydropyridine) which is a toxin specific to dopaminergic neurons, or rotenone and paraquat which affect the electron transport chain of dopaminergic neurons, etc.

The composition for fabricating a Parkinson's disease animal model of the present invention may comprise the Proteus mirabilis strain in a concentration of 1 to 1×10²⁰ CFU/ml (per animal), and preferably, the composition may comprise the Proteus mirabilis strain in a concentration of 2×10⁹ CFU/ml (per animal). The composition for fabricating a Parkinson's disease animal model of the present invention may be in any form as long as the composition can be orally administered, and preferably, the composition may be a feed composition.

According to yet another embodiment of the present invention, the present invention provides a method for fabricating a Parkinson's disease animal model, comprising administering a Proteus mirabilis strain to an animal excluding humans.

The present invention may fabricate a Parkinson's disease animal model by orally administering the composition for fabricating a Parkinson's disease animal once a day for 3 to 7 days, preferably for 5 days.

In a specific example of the present invention, a Parkinson's disease animal model was fabricated by administering the Proteus mirabilis strain to a test animal. It has been confirmed that dopaminergic neurons decreased in the corpus striatum and the substantia nigra of the brain in the Parkinson's disease animal model fabricated above as compared with the normal control group (FIG. 1), and that cranial nerve inflammation occurred, α-synuclein was accumulated, LPS increased in each tissue, and motor ability was remarkably damaged, thereby causing Parkinson's disease (FIG. 5).

In the present invention, the animal refers to an animal excluding humans, including, for example, monkeys, dogs, cats, rabbits, guinea pigs, rats, mice, cows, sheep, pigs, goats, etc., but is not limited thereto. In a specific example of the present invention, a mouse (C57BL/6) was used, but the animal is not limited thereto.

In the present invention, the Proteus mirabilis strain may be administered to the animal through a normal route without limitation as long as the composition can be delivered to the animal and show effect. For example, the composition may be administered by an injection, or formulated and orally administered. Preferably, the composition may be added to the feed to be ingested.

Further, the method for fabricating a Parkinson's disease animal model of the present invention may comprise further administering a neurotoxin causing Parkinson's disease, selected from the group consisting of 6-OHDA, MPTP, MPTP/probenecid, rotenone and paraquat, in addition to the Proteus mirabilis strain. Here, the probenecid serves as an agent of inhibiting the discharge of MPTP.

The Proteus mirabilis strain and the neurotoxin causing Parkinson's disease selected from the group consisting of 6-OHDA, MPTP, MPTP/probenecid, rotenone and paraquat may be administered to the animal sequentially, simultaneously or intermittently.

The MPTP may be administered in an amount of 1 to 1000 mg/kg. Preferably, the MPTP may be administered in an amount of 15 mg/kg when administered sequentially with the Proteus mirabilis strain or in an amount of 30 mg/kg when administered alone.

All together, the 6-OHDA may be administered in an amount of 1 μg/μl to 100 μg/μl, MPTP/probenecid may be administered in amount of 1 to 1000 mg/kg, rotenone may be administered in an amount of 1 to 500 mg/kg, and paraquat may be administered in an amount of 1 to 100 mg/kg.

In a specific example of the present invention, in order to confirm the effect of the Proteus mirabilis strain in increasing toxicity of MPTP, the motor ability of an animal model fabricated by simultaneously administering the Proteus mirabilis strain and MPTP was evaluated, and as a result, it was observed that the motor ability was remarkably damaged as compared with the case when MPTP was administered alone.

According to yet another embodiment of the present invention, the present invention provides a method for screening an agent for treating Parkinson's disease, comprising administering a candidate drug of the agent for treating Parkinson's disease to the Parkinson's disease animal model fabricated; and observing the degree of mitigation of Parkinson's disease symptoms to determine the treatment effect of the candidate drug on Parkinson's disease.

The candidate drug is a newly synthesized compound or a known compound, and includes any substance expected to show an effect in preventing or treating Parkinson's disease without limitation.

If Parkinson's disease symptoms (e.g., decrease in dopaminergic neurons, cranial nerve inflammation, increased level of LPS in each tissue, accumulation (or increase) of α-synuclein, loss (or damage) of motor ability, etc.) induced by treatment with the Proteus mirabilis strain are observed to be mitigated by administering a candidate drug of the agent for treating Parkinson's disease to the Parkinson's disease animal model of the present invention, it is determined that the corresponding drug is effective in preventing or treating Parkinson's disease.

### Advantageous Effects

The information providing method of the present invention may diagnose Parkinson's disease at an early stage using a simple method by measuring the amount of any one target selected from the group consisting of a Proteus mirabilis strain, a metabolite produced by the Proteus mirabilis strain, and α-synuclein in a biological sample of a subject and comparing the same with the amount of the target of a normal control group. Also, the Parkinson's disease animal model fabricated by administering a composition for fabricating a Parkinson's disease animal model, comprising the Proteus mirabilis strain of the present invention accompanies various symptoms associated with Parkinson's disease, and thus can be usefully used in a method for screening a candidate drug of an agent for treating Parkinson's disease, etc.

### DESCRIPTION OF DRAWINGS

FIG. 1 illustrates the immunohistochemical stain results and graphs showing the decrease of dopaminergic neurons in the corpus striatum and the substantia nigra of the brain in a Proteus mirabilis strain administration group as compared with a normal control group;

FIG. 2 illustrates the immunohistochemical stain results and graphs showing the decrease of dopaminergic neurons in the corpus striatum and the substantia nigra of the brain in an MPTP 15 mg/kg administration group, an MPTP 30 mg/kg administration group, and a Proteus mirabilis strain administration group;

FIG. 3 illustrates the drawing and graph showing that in a Proteus mirabilis strain administration group, astrocyte (GFAP-positive cells) and microglia (CD11b-positive cells) are activated in the corpus striatum and the substantia nigra of the brain so that cranial nerve inflammation occurs;

FIG. 4 illustrates the graphs showing that LPS increases in the feces and plasma over time after administering the Proteus mirabilis strain;

FIG. 5 illustrates the graphs showing that α-synuclein increases in the large intestine and the substantia nigra of the brain over time after administering the Proteus mirabilis strain;

FIG. 6 illustrates the graphs showing the pole test result and the rotarod test result performed on an MPTP 15 mg/kg administration group, an MPTP 30 mg/kg administration group, and a Proteus mirabilis strain administration group;

FIG. 7 illustrates the graphs showing the open field test result and the rotarod test result performed on a Proteus mirabilis strain administration group;

FIG. 8 illustrates the graphs showing the decrease in dopamine content and DOPAC content in the corpus striatum of the brain in a Proteus mirabilis strain administration group; and

FIG. 9 illustrates the graphs showing the measurement of the number of enterobacteria and the number of bacteria of the Proteus mirabilis strain in an MPTP/Proteus mirabilis strain administration group and an MPTP or 6-OHDA administration group.

### MODES OF THE INVENTION

Hereinafter, the present invention will be explained in more detail with reference to the examples. These examples are to explain the present invention more specifically, and do not intend to limit the scope of the present invention.

### EXAMPLES

### Example 1: Fabrication of Parkinson's Disease Animal Model

1-1. Preparation of Test Animal

A C57 black 6 (C57BL/6 test animal, Daehanbiolink, Republic of Korea) male test animal at 7 weeks of age with a body weight of 25-28 g was purchased, and supplied with sufficient feed and water to adapt to the test environment. The animal test was performed after an adjustment period of about one week.

1-2. Separation of Proteus mirabilis Strain

Fresh feces (about 0.3 g) was obtained from a Parkinson's disease animal model fabricated by intraperitoneally administering 30 mg/kg of MPTP hydrochloride to the test animal once a day for 5 days, and diluted to be cultured in a BL agar medium (for 3 days) or a DHL agar medium (for 2 days) at 37° C. under anaerobic conditions (Nissui Pharmaceutical Co., Japan).

The cultured strain was identified by using gram staining, sugar utility and 16S rRNA sequencing. As a result of confirming the base sequence (SEQ ID NO: 1) of the 16S rRNA gene of the cultured strain, it could be confirmed that the cultured strain is Proteus mirabilis.

1-3. Proteus mirabilis Strain Induced Parkinson's Disease Animal Model

A Parkinson's disease animal model was fabricated by orally administering the Proteus mirabilis strain cultured in 1-2 above to the test animal in a concentration of 2×10⁹ CFU/ml (per animal) once a day for 5 days (hereinafter, “Proteus mirabilis strain administration group”).

1-4. Fabrication of MPTP Induced Parkinson's Disease Animal Model

A Parkinson's disease animal model was fabricated by intraperitoneally administering to the test animal 15 mg/kg of MPTP hydrochloride (hereinafter, “MPTP 15 mg/kg single administration group”) or 30 mg/kg of MPTP hydrochloride (hereinafter, “MPTP 30 mg/kg single administration group”) once a day for 5 days. The control group was administered with the same amount of sterilized physiological saline by the same method.

1-5. Fabrication of MPTP/Proteus Mirabilis Strain Induced Parkinson's Disease Animal Model

In order to find out the effect of the Proteus mirabilis strain in increasing toxicity of MPTP, a Parkinson's disease animal model was fabricated by intraperitoneally administering 15 mg/kg of MPTP hydrochloride to the test animal once a day for 5 days simultaneously with orally administering the Proteus mirabilis strain in a concentration of 2×10⁹ CFU/ml (per animal) (hereinafter, “MPTP/Proteus mirabilis strain administration group”).

1-6. Fabrication of MPTP/Probenecid Induced Parkinson's Disease Animal Model

25 mg/kg of MPTP hydrochloride was intraperitoneally administered to the test animal 30 minutes after dissolving probenecid in 5% NaHCO₃ and intraperitoneally administering the same in an amount of 100 mg/kg. A Parkinson's disease animal model was induced by administering the same a total of 10 times at an interval of 3.5 days (hereinafter. “MPTP/p administration group”). The control group was administered with the same amount of sterilized physiological saline by the same method.

1-7. Fabrication of 6-OHDA (6-Hydroxydopamine) Induced Parkinson's Disease Animal Model

A Parkinson's disease animal model was fabricated by diluting 16 μg of 6-OHDA in 2 μl of 0.1% ascorbic acid, and injecting the same to the test animal once through stereotaxic injection at the corpus striatum site (AP +0.5, ML +2.0, DV −3.0 according to The Mouse Brain in Stereotaxic Coordinates second edition) at a rate of 0.5 μl/minute (hereinafter, “6-OHDA administration group:”). The control group was injected once with the same amount of sterilized physiological saline by the same method.

1-8. Fabrication of Rotenone Induced Parkinson's Disease Animal Model

A Parkinson's disease animal model was fabricated by intravenously administering to the test animal a solution in which 2 mg/kg of rotenone was dissolved in a solution diluting dimethylsulfoxide (DMSO) and polyethylene glycol (PEG) in a ratio of 1:1 using an osmotic mini pump once a day for 35 days. The control group was injected with the same amount of a 1:1 diluted solution of DMSO and PEG by the same method (Nat Neurosci. 2000 December; 3(12):1301-6).

1-9. Fabrication of Paraquat Induced Parkinson's Disease Animal Model

A Parkinson's disease animal model was fabricated by intraperitoneally administering to the test animal a solution in which 10 mg/kg of paraquat was dissolved in sterilized physiological saline a total of three times for three weeks at an interval of once a week. The control group was injected with the same amount of sterilized physiological saline by the same method (Neurobiol Dis. 2002 July; 10(2):119-27).

### Example 2: Confirmation of Likelihood of Occurrence of Parkinson's Disease by Proteus mirabilis Strain

In order to confirm the likelihood of the occurrence of Parkinson's disease by Proteus mirabilis strain, tests were performed to evaluate the damage to dopaminergic neurons, the degree of expression of inflammation, and the degree of expression of α-synuclein in the Parkinson's disease animal models fabricated in Examples 1-3 to 1-5 above.

2-1. Confirmation of Damage to Dopaminergic Neuron

In order to evaluate the degree of damage to dopaminergic neurons in the Parkinson's disease animal model, an immunohistochemical staining test was performed.

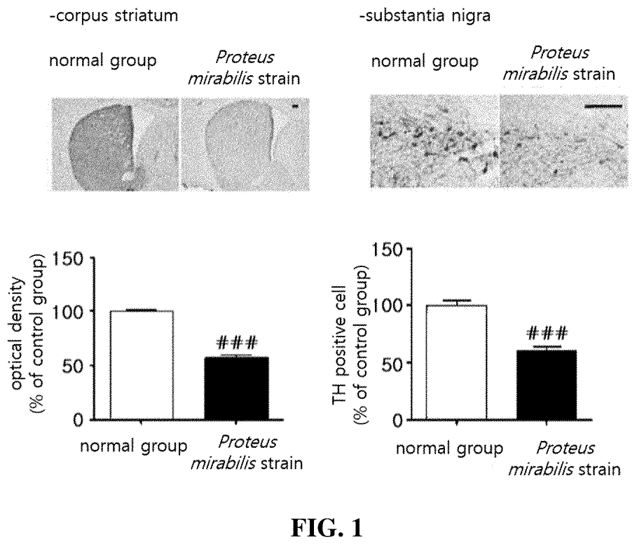

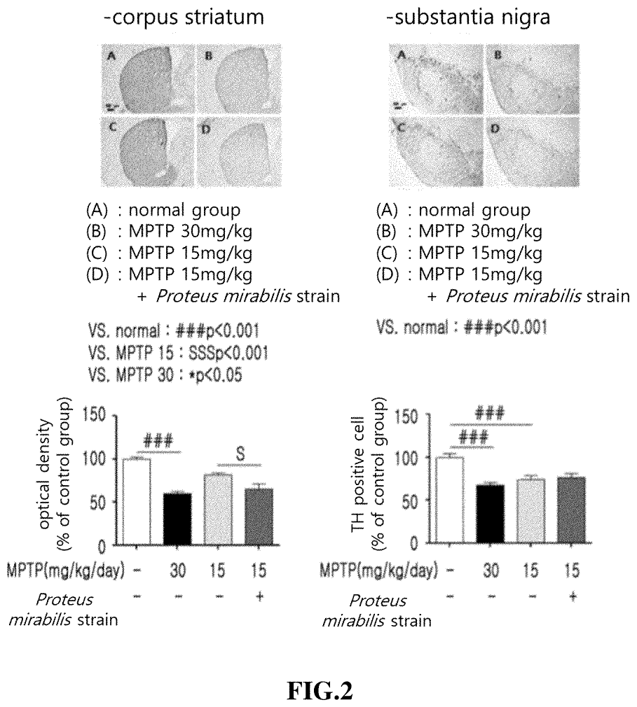

After anesthetizing each Parkinson's disease animal model fabricated according to the methods in Examples 1-3 to 1-5 above and conducting perfusion, the brain was extracted and the brain tissue was fixed with 4% PFA. Thereafter, the brain tissue subjected to a postfixation process was sliced with a thickness of 30 μm using a frozen slicer and fixed to a slide. Each tissue of the corpus striatum and the substantia nigra was immunostained using a tyrosine hydroxylase (TH) antibody, and colored using diaminobenzidine. The optical density (OD) of TH positive cells in the corpus striatum and the number of TH positive cells in the substantia nigra were counted to quantify the degree of damage to dopaminergic neurons. The results are shown in FIGS. 1 and 2.

As illustrated in FIG. 1, it has been confirmed that the dopaminergic neurons decreased in the corpus striatum and the substantia nigra of the Proteus mirabilis strain administration group as compared with the normal control group. Also, as illustrated in FIG. 2, the MPTP/Proteus mirabilis strain administration group showed a higher degree of damage to dopaminergic neurons in the corpus striatum and the substantia nigra of the brain as compared with the MPTP 15 mg/kg single administration group. It was confirmed that this is a level similar to the MPTP 30 mg/kg single administration group.

2-2. Confirmation of Expression of Cranial Nerve Inflammation

In order to confirm the expression of cranial nerve inflammation in the Proteus mirabilis strain administration group, an immunohistochemical staining test was performed.

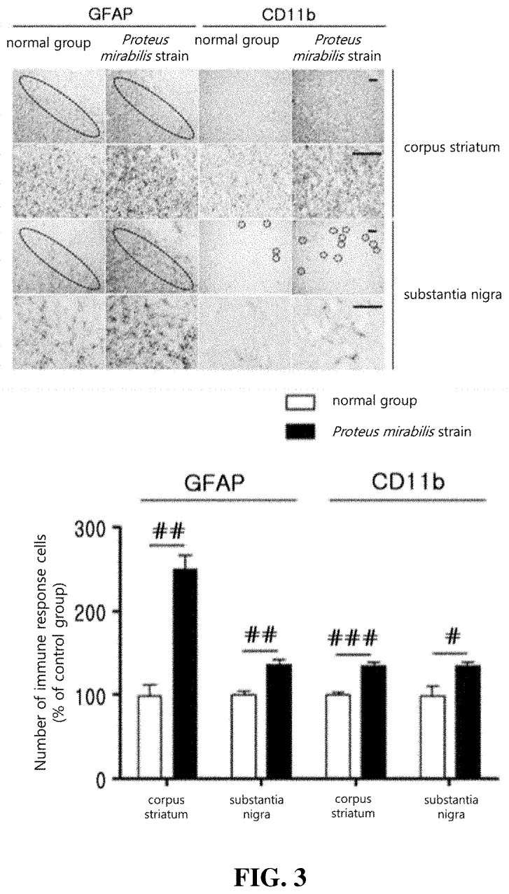

The tissues of the corpus striatum and the substantia nigra obtained from the Proteus mirabilis strain administration group were immunostained using a glial fibrillary acidic protein (GFAP) and a CD11b antibody, respectively, and colored using diaminobenzidine. The results are shown in FIG. 3.

As illustrated in FIG. 3, it has been confirmed from the activation of the astrocyte (GFAP-positive cell) and the microglia (CD11b-positive cell) in the corpus striatum and the substantia nigra of the Proteus mirabilis strain administration group that cranial nerve inflammation occurred.

Also, in order to evaluate the degree of expression of inflammation in the Proteus mirabilis strain administration group, the amount of LPS was measured.

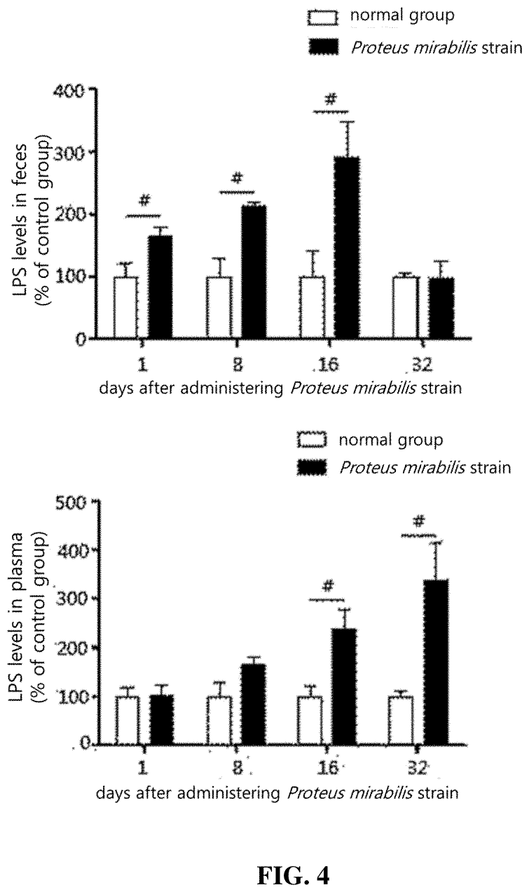

After administering the Proteus mirabilis strain to the test animal, feces were collected on the 1^{st}, 8^{th}, 16^{th} and 32^{nd} day after administration. Blood was collected 24 hours after completion of the motor ability test. From the feces and plasma, LPS was quantified according to the user manual of the LAL quantifying kit (Cape Cod Inc., Falmouth, U.S.A.). The results are shown in FIG. 4.

As illustrated in FIG. 4, it has been confirmed that the amount of LPS increased statistically significantly on the 1^{st}, 8^{th} and 16^{th} day after administering the Proteus mirabilis strain and on the 16^{th} and 32^{nd} day after administering the Proteus mirabilis strain in the feces and the plasma, respectively, as compared with the normal control group.

2-3. Confirmation of Expression of α-Synuclein

In order to evaluate the degree of expression of α-synuclein in the substantia nigra of the brain and the intestinal tissue in the Proteus mirabilis strain administration group, western blot was performed.

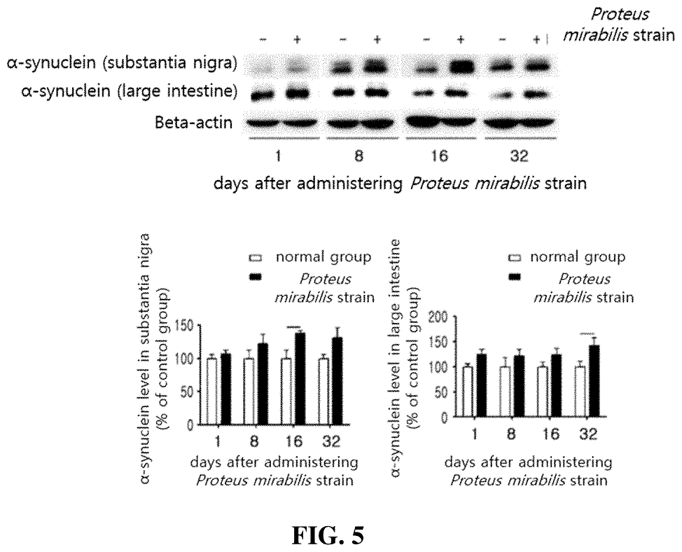

After treating the tissue of the large intestine and the substantia nigra of the brain extracted from the Proteus mirabilis strain administration group with an α-synuclein primary antibody (BD Biosciences. USA) 1:1000), the degree of expression was confirmed with LAS-4000 mini system (Fujifilm Corp., Japan), and quantified using Image J software (National Institute of Health. USA). The results are shown in FIG. 5.

As illustrated in FIG. 5, it has been confirmed that in the Proteus mirabilis strain group as compared with the normal group, the amount of α-synuclein has a tendency of increasing on the 8^{th}, 16^{th} and 32^{nd} day after administering the Proteus mirabilis strain and on the 1^{th}, 8^{th}, 16^{th} and 32^{nd} day after administering the Proteus mirabilis strain in the substantia nigra of the brain and the large intestine, respectively, as compared with the normal control group. In particular, it has been confirmed that the level increased statistically significantly on the 16^{th} day after administering the Proteus mirabilis strain and on the 32^{nd} day after administering the Proteus mirabilis strain in the substantia nigra of the brain and the large intestine, respectively, as compared with the normal control group.

2-4. Motor Ability Behavior Evaluation

In order to evaluate behavior disorder, a pole test, an open field test and a rotarod test were performed on the Parkinson's disease animal models fabricated in Examples 1-3, 1-4 and 1-5.

Specifically, for the pole test, on the 16^{th} day after single administration of 15 mg/kg of MPTP, administration of 15 mg/kg of MPTP and Proteus mirabilis strain, and single administration of 30 mg/kg of MPTP, the test animal was placed on a pole having a width of 0.8 cm at a height of 55 cm facing the sky, and then the total time required (T-LA time) for the test animal to get to the floor after being rotated was measured.

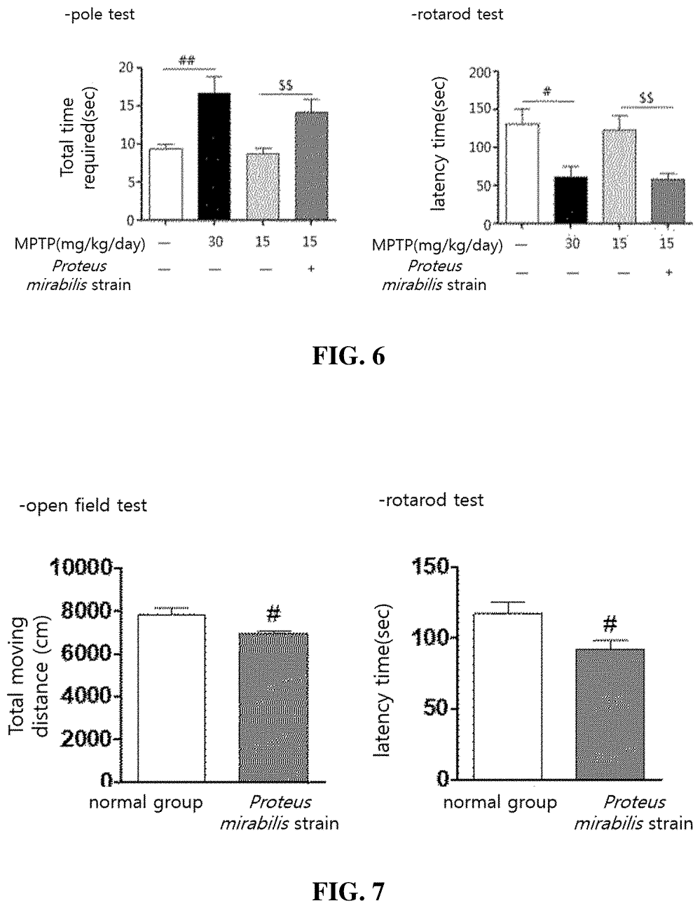

Further, in order to evaluate the motor deficit and balance maintenance of the test animal, a rotarod test was performed. The rotarod device used for the test is a rotatable cylindrical rod with five partitions having a diameter of 7 cm and a height of 60 cm at an interval of 15 cm. The latency time (sec) from the time of placing the test animal on a rod rotating at a rate of 20 rpm until the test animal falls off was measured by a rotarod. All test animals were subjected to the test after sufficient training, and the average value was set to be the latency time. The maximum measurement time was limited to 300 seconds. The results are shown in FIGS. 6 and 7.

In addition, in order to evaluate the walking activity level of the test animal, an open field test was performed. The open field test device used for the test is an acryl box with a white bottom, which has a width of 40 cm, a length of 25 cm, and a depth of 18 cm. In order to evaluate the walking activity level according to the instinctive nocturnal habit of a mouse, the test was performed between 9:00 pm and 2:00 am. The test animal was placed in the middle of the acryl box, and the total moving distance (cm) of the test animal was calculated using a viewer system (Viewer. Biobserve) for 30 minutes. The results are shown in FIG. 7.

As illustrated in FIG. 6, in the case of administering 15 mg/kg of neurotoxic substance MPTP together with the Proteus mirabilis strain, it has been confirmed from the pole test and the rotarod test that motor ability was statistically significantly damaged as compared with the MPTP 15 mg/kg single administration group, and that motor ability was damaged to a level similar to the MPTP 30 mg/kg single administration group.

Also, as illustrated in FIG. 7, in the case of the Proteus mirabilis single administration group, it has been confirmed from the open field test and the rotarod test that motor ability was statistically significantly damaged as compared with the normal group.

2-5. Measurement of the Contents of Dopamine (DA) and Dopamine Metabolite (DOPAC) in the Corpus Striatum

In order to confirm the content of dopamine (DA) and 3,4-dihydroxyphenylacetic acid (DOPAC), which is one of dopamine metabolites, in the corpus striatum of the brain, a high performance liquid chromatography (HPLC) was performed on the Parkinson's disease animal model fabricated in Example 1-3.

Specifically, after homogenizing the tissue of the corpus striatum of the brain obtained by sacrificing the test animal together with 0.2 M perchloric acid, a supernatant obtained by centrifugation (0° C. 14000×g) for 20 minutes was used as a sample, and Dionex HPLC was used as a THERMO Hypersil GOLD column (250×2.1 mm, 5 μm). As a mobile phase, 150 mM of ammonium acetate of pH 4.0, 140 μM of ethylene diamine tetra acetic acid, 15% methanol and 5% acetonitrile were used at a rate of 0.2 ml/minute. For data analysis, Chromeleon™ software (Version 6.40) was used. The contents of dopamine and dopamine metabolite were converted in comparison with the standard and quantified, and the proteins were quantified using the Bradford test method.

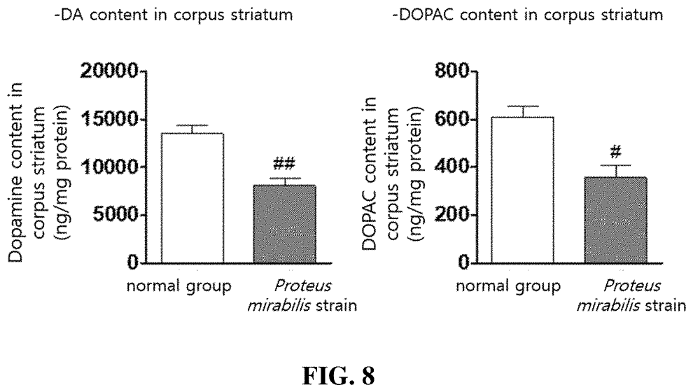

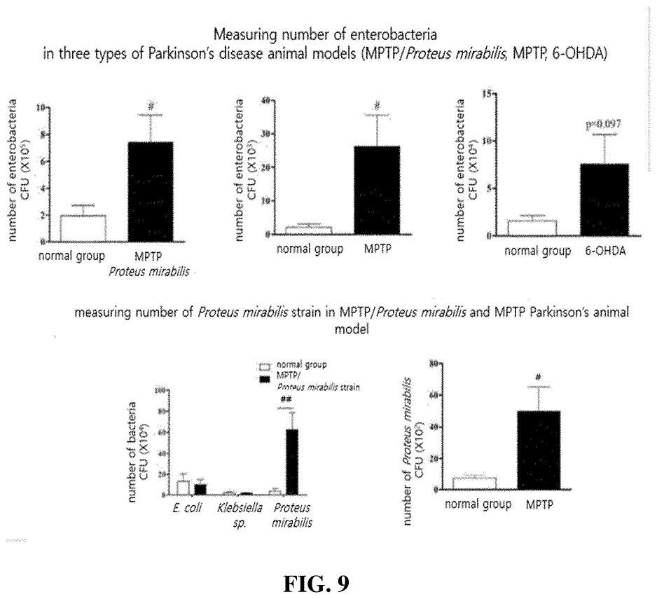

As illustrated in FIG. 8, it has been confirmed that the Proteus mirabilis strain administration group presented the reduced contents of dopamine and DOPAC in the corpus striatum, as compared with the normal control group.

Through the above tests, it could be confirmed that the Parkinson's disease animal models were fabricated by the Proteus mirabilis strain.

### Example 3: Measurement of the Number of Bacteria in Parkinson's Disease Animal Model

After collecting fresh feces from each test animal, the feces were diluted by 10 times with sterilized PBS and spread on DHL and BL agar plates. Enterobacteriaceae. E. coli. Klebsiella sp. and Proteus sp. strains grew in the DHL agar plate, and the number of bacteria was confirmed through the color and shape of colonies one day after aerobic respirational culturing. The E. coli and Klebsiella sp. strains form red colonies, and the Proteus mirabilis strain forms black colonies. The number of bacteria was measured by selecting colonies having black spots or black colonies from the colonies.

3-1. Measurement of the Number of Enterobacteria

The number of enterobacteria was measured in each of the MPTP 15 mg/kg single administration group, MPTP/p administration group and 6-OHDA administration group. The results are shown in the following table 1 and FIG. 9.

TABLE 1

| | MPTP/ | | | | |
|---|---|---|---|---|---|
| Normal | Proteus | Normal | | Normal | |
| control | mirabilis | control | | control | |
| group | strain | group | MPTP | group | 6-OHDA |
| 1.95 × 10⁵ | 7.4 × 10⁵ | 2.18 × 10³ | 26.28 × 10³ | 1.6 × 10³ | 7.56 × 10³ |

As illustrated in FIG. 9, it has been confirmed that the number of bacteria of Enterobacteria strains in the three types of Parkinson's disease animal models of the MPTP/Proteus mirabilis strain administration group, MPTP 30 mg/kg single administration group and 6-OHDA administration group was remarkably greater than that of the normal control group.

3-2. Measurement of the Number of Bacteria of Proteus mirabilis Strain

The number of bacteria of E. coli, Klebsiella sp. and Proteus mirabilis strains in the MPTP/Proteus mirabilis strain administration group was measured. The results are shown in the following table 2 and FIG. 9.

TABLE 2

| | Number of bacteria in the | Number of |
|---|---|---|
| | MPTP/Proteus mirabilis | bacteria in control |
| Strain | administration group (CFU (×10⁴) | group (CFU (×10⁴) |
| E. coli | 1.0 | 13.5 |
| Klebsiella sp. | 1.66 | 2 |
| P. mirabilis | 62.33 | 4 |

Also, the number of bacteria of the Proteus mirabilis strains in the normal control group and the MPTP 30 mg/kg administration group was measured. The results are shown in the following table 3 and FIG. 9.

TABLE 3

| | Number of bacteria of Proteus mirabilis strain |
|---|---|
| Strain | (CFU (×10²) |
| Normal control group | 7.3 |
| MPTP administration | 49.8 |
| group | |

As illustrated in FIG. 9, as a result of measuring the number of bacteria of the Proteus mirabilis strain and the number of bacteria of other strains of the Enterobacteriaceae family to which the Proteus mirabilis strain belongs, i.e., E. coli and Klebsiella sp. strains, in the Parkinson's disease animal model, and comparing the same with the normal control group, it has been confirmed that the number of bacteria of the Proteus mirabilis strain was remarkably greater than that of the normal control group, as compared with the two strains.

1. An information providing method for diagnosing Parkinson's disease, the method comprising:
a) measuring the amount of any one target selected from the group consisting of a Proteus mirabilis strain, a metabolite produced by the Proteus mirabilis strain, and α-synuclein in a biological sample of a subject; and
b) comparing the amount of the target measured in the above step a) with the amount of the target measured from a biological sample of a normal control group which is not suffering from Parkinson's disease.

2. The information providing method of claim 1, further comprising:
c) classifying, i) when the amount of the target measured in the above step a) is greater than the amount of the target measured from a biological sample of a normal control group which is not suffering from Parkinson's disease, that the subject develops or has a high risk of developing Parkinson's disease, and classifying, ii) when the amount of the target measured in the above step a) is similar or equal to the amount of the target measured from a biological sample of a normal control group which is not suffering from Parkinson's disease, that the subject does not develop Parkinson's disease.

3. The information providing method of claim 1, wherein the biological sample is tissue, blood, whole blood, serum, plasma, saliva, sputum, cerebrospinal fluid, urine, colonic tissue or feces.

4. The information providing method of claim 1, wherein the metabolite produced by the Proteus mirabilis strain is lipopolysaccharide (LPS).

5. A composition for fabricating a Parkinson's disease animal model, comprising a Proteus mirabilis strain as an active ingredient.

6. The composition of claim 5, wherein the composition comprises the Proteus mirabilis strain in a concentration of 1 to 1×1020 CFU/ml (per animal).

7. The composition of claim 6, wherein the composition comprises the Proteus mirabilis strain in a concentration of 2×109 CFU/ml (per animal).

8. The composition of claim 5, wherein the composition is orally administered once a day for 3 to 7 days.

9. The composition of claim 5, wherein the composition is a feed composition.

10. A method for fabricating a Parkinson's disease animal model, the method comprising: administering a Proteus mirabilis strain to an animal excluding humans.

11. The method of claim 10, the method comprising: further administering a neurotoxin causing Parkinson's disease, selected from the group consisting of 6-OHDA (6-hydroxydopamine), MPTP (1-methyl-4-phenyl-1,2,4,6-tetrahydropyridine), MPTP/probenecid, rotenone and paraquat.

12. The method of claim 11, wherein the Proteus mirabilis strain and the neurotoxin causing Parkinson's disease are administered to the animal sequentially, simultaneously or intermittently.

13. The method of claim 11, wherein the neurotoxin causing Parkinson's disease is MPTP.

14. The method of claim 13, wherein the MPTP is administered in an amount of 1 to 1000 mg/kg.

15. The method of claim 14, wherein the MPTP is administered in an amount of 15 mg/kg when administered sequentially with the Proteus mirabilis strain.

16. A Parkinson's disease animal model fabricated by the method of claim 10.

17. A method for screening an agent for treating Parkinson's disease, the method comprising:
administering a candidate drug of the agent for treating Parkinson's disease to the Parkinson's disease animal model of claim 16; and observing the degree of mitigation of Parkinson's disease symptoms to determine the treatment effect of the candidate drug on Parkinson's disease.

18. The Parkinson's disease animal model of claim 16, wherein the Parkinson's disease animal model further comprises a neurotoxin causing Parkinson's disease, selected from the group consisting of 6-OHDA (6-hydroxydopamine), MPTP (1-methyl-4-phenyl-1,2,4,6-tetrahydropyridine), MPTP/probenecid, rotenone and paraquat.

19. The method of claim 17, wherein the Parkinson's disease animal model further comprises a neurotoxin causing Parkinson's disease, selected from the group consisting of 6-OHDA (6-hydroxydopamine), MPTP (1-methyl-4-phenyl-1,2,4,6-tetrahydropyridine), MPTP/probenecid, rotenone and paraquat.
